(19) United States
(12) Patent Application Publication
(10) Pub. No.: US 2022/0218715 A1
(43) Pub. Date: Jul. 14, 2022

(54) NOVEL USE OF PYRROLO-PYRIDINE DERIVATIVE COMPOUND FOR PREVENTION AND/OR TREATMENT OF CANCER

(71) Applicant: VORONOI CO., LTD., Incheon (KR)

(72) Inventors: Jae Young AHN, lncheon (KR); Hwan KIM, Seoul (KR); Dong Hyuk SEO, Incheon (KR); Ji Eun CHOI, Seoul (KR); Hye Min HWANG, Gyeonggi-do (KR); Hyun Kyung KIM, Incheon (KR); Sung Hwan KIM, lncheon (KR); Dae Kwon KIM, Daegu (KR)

(21) Appl. No.: 17/595,655

(22) PCT Filed: May 22, 2020
(86) PCT No.: PCT/KR2020/006730

§ 371 Date: Nov. 22, 2021

(30) Foreign Application Priority Data
May 22, 2019 (KR) 10-2019-0060297

(51) Int. Cl. A61K 31/5377; A61K 45/06; A61P 35/00

(52) U.S. Cl. CPC A61K 31/5377; A61P 35/00; A61K 45/06

(57) ABSTRACT

The present invention relates to a pharmaceutical composition for the prevention or treatment of cancer, comprising an anticancer drug and a pyrrolo-pyridine derivative as an active ingredient. A compound represented by Chemical Formula I according to the present invention, an isomer thereof, or a pharmaceutically acceptable salt thereof has excellent inhibitory activity against protein kinases, and thus, when used in combination with other anticancer drugs, can be effectively used in the prevention, treatment or amelioration of cancer.

### TECHNICAL FIELD

The present invention relates to a novel use of a pyrrolo-pyridine derivative compound for prevention and/or treatment of cancer.

### BACKGROUND ART

Epidermal growth factor receptor (EGFR) is a transmembrane protein expressed on the surface of epidermal cells, and belongs to the group of tyrosine kinases which are cell cycle regulatory factors.

It is activated when EGF or TGF-α binds to the extracellular domain, and induces a self-phosphorylation reaction to promote cell proliferation and growth. If a genetic modification which impacts the function of such growth factor receptor or causes overexpression of receptor and ligands occurs, cancer may be induced.

Further, EGFR is a protein product of the oncogene ErbB or ErbB1. ErbB or ErbB1 is one of the ERBB group of protooncogenes, which are known as important factors in the occurrence of cancer. Results are reported wherein EGFR expression is increased in lung cancer, head and neck cancer, breast cancer, bladder cancer, stomach cancer, and the like. The ERBB oncogene group encodes four structurally related transmembrane receptors, that is, EGFR, HER-2/neu(ErbB2), HER-3 (ErbB3), and HER-4 (ErbB4).

To treat lung cancer, head and neck cancer, breast cancer, bladder cancer, stomach cancer, and the like, various EGFR-targeting drugs have been developed, and representative drugs include Gefitinib (AstraZeneca UK Ltd., trademark “IRESSA”) and Erlotinib (Genentech, Inc. & OSI Pharmaceuticals, Inc., trademark “TARCEVA”). Gefitinib and Erlotinib are quinazoline compounds, and inhibit cell growth by inhibiting the tyrosine kinase activity of EGFR to suppress phosphorylation.

Despite these targeted drugs being very effective drugs, problems are reported such as not showing a measurable reaction due to individual differences among patients or exhibiting resistance to therapeutic effects with prolonged use. In fact, only around 10% of non-small-cell lung cancer patients are reported as exhibiting reaction to these drugs. Accordingly, studies on concomitant therapies to supplement the problems of EGFR targeting drugs are being carried out, and whereas various compounds which may be used concomitantly with EGFR are known, they are reported as also having problem such as cytotoxicity when used concomitantly.

To resolve such limitations of targeted anticancer drugs, there is active ongoing development of cancer immunotherapy drugs which have few adverse reaction and resistance problems, and where, even if administration is discontinued, immune cells remember cancer cells and continue to attack cancer cells. Cancer immunotherapy drugs can be categorized broadly as immune checkpoint inhibitors, immune cell therapy agents, therapeutic antibodies, and anticancer vaccines. Of these, immune check point inhibitors are drugs which block activity of immune checkpoint proteins involved in T cell suppression, thereby activating T cells to attack cancer cells. Representatively, these use antibodies recognizing CTLA-4, PD-1, and PD-L1.

The present inventors, by setting their attention to various pyrrolo-pyridine derivatives reported as exhibiting activity as inhibitors with regard to various protein kinases (Korean Laid-open Patents No. 10-2018,0015142, No. 10-2017-0106452, and No. 10-2017-0058465), have identified pyrrolo-pyridine derivatives which, when used concomitantly with EGFR target drugs and/or immune checkpoint inhibitors, do not have cytotoxicity and can vastly improve the anticancer effect of a single drug, have completed pharmaceutical compositions for prevention or treatment of cancer combining an EGFR targeting drug and/or immune checkpoint inhibitor with pyrrolo-pyridine derivative compounds.

### DETAILED DESCRIPTION OF THE INVENTION

### Technical Problem

A purpose of the present invention is to provide a pharmaceutical composition for prevention and/or treatment of cancer, the pharmaceutical composition comprising an EGFR targeting drug and/or immune checkpoint inhibitor; and a pyrrolo-pyridine derivative compound, an isomer of the same, or pharmaceutically acceptable salt of the same, as an active ingredient.

Another purpose of the present invention is to provide a pharmaceutical composition for prevention and/or treatment of cancer, the pharmaceutical composition comprising a pyrrolo-pyridine derivative compound, an isomer of the same, or pharmaceutically acceptable salt of the same as an active ingredient, for concomitant administration with an EGFR targeting drug and/or immune checkpoint inhibitor.

Yet another purpose of the present invention is to provide a method for prevention and/or treatment of cancer by administering to a subject an effective dose of EGFR targeting drug and/or immune checkpoint inhibitor; and a pyrrolo-pyridine derivative compound, an isomer of the same, or pharmaceutically acceptable salt of the same.

Yet another purpose of the present invention is to provide a kit for prevention and/or treatment of cancer, the kit comprising an EGFR targeting drug and/or immune checkpoint inhibitor; and a pyrrolo-pyridine derivative compound, an isomer of the same, or pharmaceutically acceptable salt of the same.

Yet another purpose of the present invention is to provide a use of a compound, an isomer of the same, or pharmaceutically acceptable salt of the same for concomitant use with an EGFR targeting drug and/or immune check point inhibitor for prevention and/or treatment of cancer.

### Technical Solution

The present invention provides a pharmaceutical composition comprising a compound represented by Chemical Formula 1, an isomer of the same or a pharmaceutically acceptable salt of the same; and a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) as active ingredients.

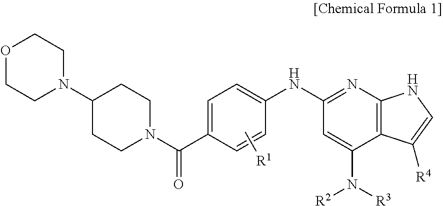

(Where, in Chemical Formula 1,

R¹ is C₁-C₃ alkoxy;

R² and R³ are each independently hydrogen, straight chain or branched chain C₁-C₁₀ alkyl, or C₃-C₆ cycloalkyl; and

R⁴ is haloalkyl).

The present invention provides a pharmaceutical composition for prevention and/or treatment of cancer, the pharmaceutical composition comprising a pyrrolo-pyridine derivative compound, an isomer of the same, or pharmaceutically acceptable salt of the same as an active ingredient, for concomitant administration with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4).

The present invention provides a method for prevention and/or treatment of cancer by administering to a subject an effective dose of a pharmaceutical composition comprising a compound represented by Chemical Formula 1, an isomer of the same or a pharmaceutically acceptable salt of the same; and a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) as active ingredients.

The present invention provides a kit for prevention and/or treatment of cancer, the kit comprising a pharmaceutical composition comprising a compound represented by Chemical Formula 1, an isomer of the same or a pharmaceutically acceptable salt of the same; and a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) as active ingredients.

The present invention provides a use of a pyrrolo-pyridine derivative compound, an isomer of the same, or pharmaceutically acceptable salt of the same, for concomitant use with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4), for prevention and/or treatment of cancer.

### Benefit(s) of the Invention

The compound represented by Chemical Formula 1, an isomer of the same or a pharmaceutically acceptable salt of the same according to the present invention, having excellent inhibitory activity against various protein kinases including DYRK1A and CLK and excellent in vivo stability, substantially increases the therapeutic effect of a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) when used concomitantly with target drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4), and is able to reduce resistance to such target drugs in cancer cells, and is able to effectively treat or prevent cancer.

### BRIEF DESCRIPTION OF THE DRAWINGS

FIG. 1 shows the results of comparing the lung cell colony formation suppression effect of treating lung cancer cell lines (PC-9 and H1975) concomitantly with Compound 1 and an EGFR target drug (Gefitinib or Osimertinib) against cases treated singularly with each substance.

FIG. 2 shows the results of comparing the expression of DYRK1A in a non-small cell lung cancer cell line and non-small cell lung cancer cell lines having resistance to Gefitinib.

FIG. 3 shows the results of examining anticancer effect for cases when a mouse model (Balb/c Nude Mice) having PC-9 lung cancer cells is administered singularly with Compound I and Osimertinib, respectively, and concomitantly.

FIG. 4 shows the results of examining anticancer effect for cases when a mouse model (Balb/c Nude Mice) having PC-9 lung cancer cells is administered singularly with Compound I and Gefitinib, respectively, and concomitantly.

FIG. 5 shows the results of examining anticancer effect for cases when a mouse model (Balb/c Nude Mice) having NCI-H1975 lung cancer cells is administered singularly with Compound I and Osimertinib, respectively, and concomitantly.

FIG. 6 shows the results of examining anticancer effect when Compound I is administered singularly to a mouse model with heteroplastic transplantation of lung cancer, colon cancer and breast cancer (FIG. 6a), and the anticancer effect against lung cancer (FIG. 6b) and colon cancer (FIG. 6c) when Compound 1 is concomitantly administered with antibody recognizing PD-1, PD-L1 and CTLA-4.

FIG. 7 shows results of measuring EGFR expression after treatment of Ba/F3 EGFR L858R/T790M/C797S, NCI-H2228 and CAL-27 cell lines with Compound 1.

FIG. 8 shows results of examining the [cancer] cell inhibition effect when Ba/F3 EGFR L858R/T790M/C797S cells are concomitantly administered Compound 1 and Osimertinib.

### BEST MODE(S) FOR CARRYING OUT THE INVENTION

Meanwhile, embodiments of the present invention may be modified into various different forms, and the scope of the present invention is not limited to the embodiments described in the following. Further, the embodiments of the present invention are provided to more fully explain the present invention to a person having ordinary skill in the art. Further, throughout the specification, to “comprise” a certain component element does not mean that, unless specifically stated to the contrary, other component elements are excluded, but that other component elements may be further comprised.

In the present specification, “halogen” may be F, Cl, Br or I.

In the present specification, “haloalkyl’ may be a straight chain or branched chain alkyl (hydrocarbon) having a carbon atom substituted with at least one atom of halogen as defined in the present specification. Non-limiting examples of haloalkyl include a methyl, ethyl, propyl, isopropyl, isobutyl and N-butyl independently substituted by F, Cl, Br or I.

In the present specification, “alkyl” may refer to a straight chain or branched chain non-cyclic saturated hydrocarbon made up of carbon atoms. Representative examples of —(C₁₋₈alkyl) may include -methyl, -ethyl, —N-propyl, —N-butyl, —N-pentyl, —N-hexyl, —N-heptyl, and N-octyl; and branched chain saturated alkyls may include -isopropyl, -sec-butyl, -isobutyl, -tert-butyl, -isopentyl, -2-methylpentyl, 3-methylpentyl, 4-methylpentyl, 2,3-dimethylbutyl, and the like. —(C₁₋₈alkyl) may be substituted or unsubstituted. For example, a C₁₋₈ alkyl group may be substituted with a phenyl to form a benzyl group.

In the present specification, “cycloalkyl” may refer to a nonaromatic saturated or unsaturated carbon ring. Non-limiting representative examples of cycloalkyl include cyclopropyl, cyclobutyl, cyclopentyl, cyclopentadienyl, cyclohexyl, cyclohexenyl, 1,3-cyclohexadienyl, 1,4-cyclophexadienyl, cycloheptyl, 1,3-cycloheptadienyl, 1,3,5-cycloheptatrienyl, cyclooctyl and cyclooctadienyl. The cycloalkyl group may be substituted or unsubstituted, in one embodiment, the cycloalkyl group may be a C₃₋₈ cycloalkyl group. A cycloakyl group of C₇ or higher may have two or more ring structures, and a specific example thereof may be a bicycloalkyl group. More specifically, bicycloheptane may be used in the present invention.

At least one of the homogeneous or heterogeneous substituents mentioned above may be substituted at like or different positions, and these may also be substituted sequentially. Here, “sequentially” refers to, in a chemical formula, one substituent being substituted followed by sequential substitution of another substituent, and, for example, in a case wherein an alkyl group is substituted, a cycloalkyl group is substituted at the alkyl group and then the cycloalkyl group is sequentially substituted by a carbonyl group, the compound may be indicated as sequentially substituted by naming the compound carbonylcycloalkylalkyl.

The present invention provides a pharmaceutical composition comprising a compound represented by Chemical Formula 1, an isomer of the same or a pharmaceutically acceptable salt of the same; and a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) as active ingredients.

1 [Chemical Formula 1]

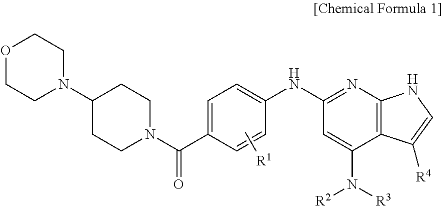

In Chemical Formula 1,

R¹ may be C₁-C₃ alkoxy;

R² and R³ may each independently be hydrogen, straight chain or branched chain C₁-C₁₀ alkyl, or C₃-C₆ cycloalkyl; and

R⁴ may be haloalkyl.

In one specific embodiment, the R¹ may be methoxy; the R² may be straight chain or branched chain C₁-C₅ alkyl, or C₃-C₄ cycloalkyl; the R³ may be hydrogen; and R⁴ may be trifluoromethyl.

In another specific embodiment, the compound represented by Chemical Formula 1 may be (4-((4-(ethylamino)-3-(trifluoromethyl)-1H-pyrrolo[2,3-b]pyridine-6-yl)amino)-3-methoxyphenyl) (4-morpholinopiperidine-1-yl)methanone.

In the present specification, a pharmaceutically acceptable salt of a compound represented by Chemical formula 1 may be interpreted as being able to exist in any form selected from a group comprising any crystalline and amorphous form, or hydrates, solvates and co-crystals of the same.

In the present specification, “hydrate” may refer to the compound of the present invention or a salt thereof, comprising a stoichiometric or non-stoichiometric amount of water bonded by a non-covalent intermolecular force. A hydrate of the compound of the present invention represented by Chemical Formula 1 may comprise a stoichiometric or non-stoichiometric amount of water bonded by a non-covalent intermolecular force. The hydrate may comprise at least one equivalents of water, preferably one to five equivalents. Such hydrate may be prepared by crystallizing, from water or solvent comprising water, the compound of the present invention represented by Chemical Formula 1, isomers of the same, or pharmaceutically acceptable salts thereof. The term “solvate” may refer to the compound of the present invention or a salt thereof, comprising a stoichiometric or non-stoichiometric amount of solvent which is bonded by a non-covalent intermolecular force. Preferred solvents include volatile solvents, non-volatile solvents, and/or solvents suitable for administration to humans. The term “isomer” refers to the compound of the present invention or a salt thereof which has an identical chemical or molecular formula but which is structurally or three-dimensionally different. Included among such isomers are structural isomers such as tautomers, R or S isomers having asymmetric carbon centers, stereoisomers including geometric isomers (trans, cis), and enantiomers. All such isomers and compounds thereof are included in the scope of the present invention. Unless otherwise explained, the solid line bonds (-) to an asymmetric carbon atom may include solid wedge ( [symbol] ) or dashed wedge ( [symbol] ) bonds which represent the absolute three-dimensional configuration.

In the present specification, the term “target drug for EGFR (epidermal growth factor receptor), PD-1 (Programmed cell death protein 1), PD-L1 (Programmed death-ligand 1) and CTLA-4 (cytotoxic T-lymphocyte-associated protein 4)” may refer to an inhibitor for EGFR, PD-1, PD-L1 and CTLA-4. In particular, PD-1, PD-L1 and CTLA-4 are proteins involved in immune checkpoints, and inhibitors for such proteins are referred to as immune checkpoint inhibitors. The target drugs may be inhibitors effective in preventing and/or treating cancer, including, but not limited to, all compounds approved for cancer treatment, compounds in clinical trials for cancer therapy, compounds demonstrating efficacy in treating cancer in mammalian subjects (for example, mouse, rat, dog, monkey, human), and compounds demonstrating efficacy against cancer cells in vitro. Many compounds exhibiting such effects are known Lee C C, et al (2014) Small-molecule EGFR tyrosine kinase inhibitors for the treatment of cancer, Expert Opinion on Investigational Drugs 23, 1333-1348).

Specifically, the EGFR target drug may be EGFR tyrosine kinase inhibitor (EGFR TKI). For example, it may be at least one of Brigatinib, CUDC-101, Erlotinib, Gepitinib, Icotinib, Lapatinib, Safitinib, Vandetanib, Baratinib, Tecevatinib, Tirpostin AG 1478, AZD3759, MTKi-327 (JNJ-26483327), Afatinib, Olmutinib (HM61713), Canertinib, CL-387785 (EKI-785), CNX-2006), Dacomitinib, Naquotinib (ASP8273), Neratinib, Osimertinib, PD168393, Pelitinib, Poziotinib, Rociletinib, TAK285, WZ4002, Alitinib (ALS-1306; AST-1306), AV-412 (MP-412), Nazartinib (EGF816) and Pyrotinib. Other EGFR targeting drugs may be EGFR antibodies, specifically Cetuximab (Erbitux®) and Panitumumab (Vectibix®).

Examples of PD-1 targeting drug include the anti PD-1 antibodies Pembrolizumab and Nivolumab, and non-limiting examples of PD-L1 targeting drugs include Atezolizumab, Avelumab and Duvalumab. An example of a CTLA-4 targeting drug may be the anti CTLA-4 antibody Ipilimumab.

The compound according to the present invention, isomers of the same, or pharmaceutically acceptable salts of the same may act as inhibitors for a protein kinase.

The protein kinase may be at least one of ALK, ALK (C1156Y), ALK (L1196M), CLK1, CLK2, CLK3, CLK4, CSNK1D, DYRK1A, DYRK1B, DYRK2, GAK, JNK1, LRRK2 (G2019S), LTK, MYLK, PAK2, PHKG1, PHKG2, STK33, ABL1-nonphosphorylated, CAMK2D, CAMKK2, CHEK2, CSNK1A1, CSNK1E, ERK5, HUNK, INSR, JAK1 (JH2domain-pseudokinase), JNK2, JNK3, LRRK2, MAPKAPK2, PLK4, and STK39.

Further, the present invention provides a pharmaceutical composition for prevention and/or treatment of cancer, the pharmaceutical composition comprising a pyrrolo-pyridine derivative compound, an isomer of the same, or pharmaceutically acceptable salt of the same as an active ingredient, and characterized in that it is administered concomitantly with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4).

In the treatment of cancer, according to various previous therapeutic methods, concomitant or combined drug therapy may result in improved results such as improved survival rate, reduced severity, delayed or elimination of relapse, or reduced adverse reaction to primary therapy (that is, an EGFR targeting drug). Further, in such concomitant therapy, drugs may be administered at smaller doses than in single drug therapy, and/or for shorter periods. That is, therapeutic effect can be maximized even with low doses and/or shorts administration periods.

In one specific embodiment of the present invention, in cases where a compound according to the present invention, an isomer of the same, or a pharmaceutically acceptable salt of the same was applied to various cancer cells concomitantly with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4), it was found that growth of the cancer cell line was effectively inhibited in a shorter time than when a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) was administered singularly, and that cell resistance to the drug was also reduced.

In the present specification, “cancer” may be, for example, leukemia, lymphoma, sarcoma, brain cancer, brain tumor, benign stromocytoma, malignant stromocytoma, pituitary cyanoma, meningioma, brain lymphoma, oligodendroglioma, craniopharyngioma, ependymoma, brain stem tumor, head and neck cancer, laryngeal cancer, oropharyngeal cancer, nasal cavity/PNS tumor, nasopharyngeal tumor, salivary gland tumor, hypopharyngeal cancer, thyroid cancer, oral cavity tumor, chest tumor, small cell lung cancer, non-small cell lung cancer, thymoma, thyroid cancer, mediastinal tumor, esophageal tumor, breast cancer, male breast cancer, abdomen-pelvis tumor, stomach cancer, hepatoma, gall bladder cancer, biliary tract tumor, pancreatic cancer, small intestinal tumor, larges intestinal tumor, colorectal cancer, anal cancer, bladder cancer, renal cell carcinoma, male genital cancer, penile cancer, prostatic cancer, female genital cancer, uterine cancer, cervical cancer, endometrial cancer, uterine sarcoma, vaginal cancer, vulva cancer, urethral cancer and skin cancer. In a specific embodiment of the present invention, the cancer may be lung cancer, non-small cell lung cancer pancreatic cancer and head and neck cancer.

In the present invention, the term “treatment” or “therapeutic” includes the suppression, delay, identification, alleviation, weakening, limiting, reduction, inhibition, avoidance or healing of illnesses, conditions, disabilities, damage or health problems, or the occurrence or progression of such statuses and/or symptoms of such statuses.

The term “prevention” refers to avoidance or reduction of risk of contracting, experiencing, suffering or having illnesses, conditions, disabilities, damage or health problems, or the occurrence or progression of such statuses and/or symptoms of such statuses.

The treatment or prevention of the disease, condition, disability, damage or health problem may be complete or partial.

The pharmaceutical composition of the present invention, by further comprising excipients, disintegrating agents, sweetening agents, lubricants and flavoring agents and the like, may be formulated using ordinary methods into tablets, capsules, powders, granules, suspensions, emulsions, syrups, or other liquid formulations.

For example, as the pharmaceutical composition of the present invention may act systemically and/or locally, and may be administered to subjects orally or non-orally, that is, through various pathways such as pulmonary route administration, intranasal administration, sublingual administration, lingual administration, buccolingual administration, rectal administration, dermal administration, transdermal administration, or conjunctival administration, [the pharmaceutical composition] may be formulated into a form suitable for the administration pathway.

For example, dosage forms suitable for oral administration are formulations comprising a compound of the present invention in crystalline and/or amorphous and/or dissolved form, and may be, for example, tablets (coated or non-coated tablets, for example using gastric fluid-resistant, delayed-dissolution or insoluble coatings), tablets or films/oblates which rapidly disintegrate in the oral cavity, films/freeze-dried preparations, capsules (for example, hard or soft gelatin capsules), sugar-coated tablets, chewables (for example, soft chewables), granules, pellets, powders, emulsions, suspensions, aerosols or solutions.

Non-oral administration may be achieved by avoiding the absorption stage (for example, through intravenous, intraarterial, intra cardiac, intrathecal or intra lumbar administration) or including absorption (for example, through intramuscular, dermal, intradermal, subdermal, percutaneous or intraperitoneal pathways).

Dosage forms suitable for non-oral administration may include solutions, suspensions, emulsions, freeze-dried preparations or preparations in the form of sterilized powders for injection.

Further, the present invention provides a kit for prevention and/or treatment of cancer, the kit comprising a pharmaceutical composition comprising a compound represented by Chemical Formula 1, an isomer of the same or a pharmaceutically acceptable salt of the same; and a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4) as active ingredients.

Further, the present invention provides a method for treatment and/or prevention of cancer, the method comprising a step of administering a pharmaceutically effective dose of the above-described compound to a subject requiring treatment and/or prevention of cancer.

In the present specification, the term “administration” refers to using a suitable method to introduce the pharmaceutical composition of the present invention to a subject suspected to have inflammatory disease, and administration may be performed through various pathways so long as the target tissue can be reached. In the present specification, “pharmaceutically effective dose” refers to a dose sufficient to treat disease with a reasonable benefit/risk ratio applicable to medical treatment, and the level of the effective dose may be decided depending on factors including the type of subject, severity of disease, age, sex, type of disease, activity of the drug, drug sensitivity, duration of administration, administration pathway and excretion rate, duration of therapy and concomitantly used drugs, and other factors known well to the medical art. The composition of the present invention may be administered as a single drug or concomitantly with other drugs, and may be administered sequentially or simultaneously with commercially marketed therapeutic agents. Further [the composition] may be administered as a single dose or multiple dose. It is important to administer a dose able to achieve maximum effect with minimum amount without adverse effects in consideration of all of the above factors, and the dose may be readily decided by a PHOSITA. The administration dose of the pharmaceutical composition of the present invention may be decided by an expert according to various factors such as patient status, age, gender and complications. As the active ingredient of the pharmaceutical composition of the present invention has excellent safety, it may be used at above the decided administration dose.

Further, the present invention provides a use of a pyrrolo-pyridine derivative compound, an isomer of the same, or pharmaceutically acceptable salt of the same, for concomitant use with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4), for prevention and/or treatment of cancer. Specifically, the present invention provides a use of a compound represented by Chemical Formula 1, an isomer of the same, or pharmaceutically acceptable salt of the same, for concomitant use with a targeting drug for at least one of EGFR (epidermal growth factor receptor), PD-1 (Programmed cell death protein 1), PD-L1 (Programmed death-ligand 1) and CTLA-4 (cytotoxic T-lymphocyte-associated protein 4), for prevention and/or treatment of cancer.

In the following, the present invention will be described in further detail with reference to embodiments and experimental examples.

Provided, that the following embodiments and experimental examples are meant only to exemplify the present invention, and the scope of the present invention is not limited to the following embodiments and experimental examples.

### <Embodiment 1> Preparation of the Compounds According to the Present Invention

The compounds of the present invention were prepared according to the preparation method for the compounds states in Korean Registered Patent Gazette No. 10-1896568. The chemical structures, names and H1 NMR data of the compounds (1 to 5) of the present invention are shown in Table 1 below.

TABLE 1

| Com- | Structure of the | | 1H NMR; |
|---|---|---|---|
| pound | Compound | Name of Compound | MS (ESI) m/z |
| 1 | 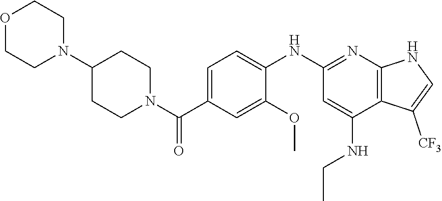 [SMILES: CCNc1cc(Nc2ccc(C(=O)N3CCC(N4CCOCC4)CC3)cc2OC)nc2[nH]cc(C(F)(F)F)c12] | (4-((4-(ethylamino)-3- (trifluoromethyl)-1H-pyrrolo[2,3- b]pyridine-6-yl)amino)-3- methoxyphenyl) (4-morpholinopiperidine- 1-yl)methanone | ¹H NMR (400 MHz, HCl salt, DMSO) δ 11.99 (s, 1H), 11.29 (s, 1H), 9.01 (br s, 1H), 8.20 (br s, 1H), 7.59 (s, 1H), 7.11 (s, 1H), 7.02 (d, J = 8.1 Hz, 1H), 6.12 (s, 1H), 5.41 (br s, 1H), 4.59- 3.91 (m, 4H), 3.89 (s, 3H), 3.87-3.80 (m, 2H), 3.38-3.19 (m, 5H), 3.15-2.79 (m, 4H), 2.25-2.12 (m, 2H), 1.76-1.66 (m, 2H), 1.25 (d, J = 7.1 Hz, 3H); 547 [M + H] ⁺ |
| 2 | 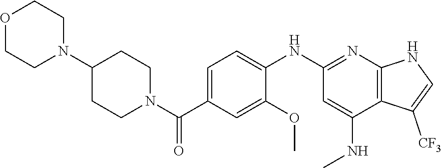 [SMILES: CNc1cc(Nc2ccc(C(=O)N3CCC(N4CCOCC4)CC3)cc2OC)nc2[nH]cc(C(F)(F)F)c12] | (3-methoxy-4-((4-(methylamino)-3- (trifluoromethyl)-1H-pyrrolo[2,3- b]pyridine-6-yl)amino)phenyl) (4- morpholinopiperidine-1-yl) methanone | ¹H NMR (400 MHz, TFA salt, Methanol-d4) δ 7.55 7.46 (m, 2H), 7.23 (d, J = 1.4 Hz, 1H), 7.13 (dd, J = 8.0, 1.5 Hz, 1H), 5.94 (s, 1H), 4.17 4.00 (m, 2H), 3.93 (s, 3H), 3.87 3.75 (m, 2H), 3.63 3.44 (m, 4H), 3.30 3.15 (m, 4H), 3.04 (s, 3H), 3.02 2.85 (m, 1H), 2.36 2.13 (m, 2H), 1.85 1.70 (m, 2H); 533 [M + H] ⁺ |
| 3 | 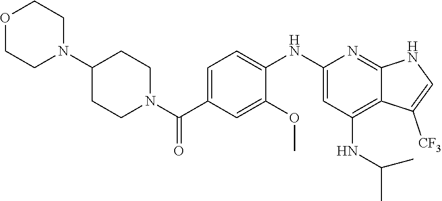 [SMILES: COc1cc(C(=O)N2CCC(N3CCOCC3)CC2)ccc1Nc1cc(NC(C)C)c2c(C(F)(F)F)c[nH]c2n1] | (4-(4-(isopropylamino)-3- (trifluoromethyl)-1H-pyrrolo[2,3- b]pyridine-6-ylamino)-3- methoxyphenyl) (4-morpholinopiperidine- 1-yl)methanone | ¹H NMR (400 MHz, TFA salt, MeOD-d4) δ 7.54-7.52 (m, 2H), 7.23 (s, 1H), 7.14 (d, J = 8.04 Hz, 1H), 5.98 (s, 1H), 4.09- 3.98 (m, 3H), 3.93 (s, 3H), 3.89-3.84 (m, 3H), 3.55-3.40 (m, 4H), 3.33-3.13 (m, 4H), 2.24-2.15 (m, 2H), 1.77-1.74 (m, 2H), 1.35 (d, J = 6.28 Hz, 6H); 561 [M + H] ⁺ |
| 4 | 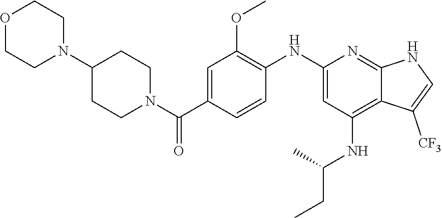 [SMILES: CC[C@H](C)Nc1cc(Nc2ccc(C(=O)N3CCC(N4CCOCC4)CC3)cc2OC)nc2[nH]cc(C(F)(F)F)c12] | (S)-(4-((4-(2-butylamino)-3- (trifluoromethyl)-1H-pyrrolo[2,3- b]pyridine-6-yl)amino)-3- methoxyphenyl) (4-morpholinopiperidine- 1-y1)-methanone | ¹H NMR (400 MHz, TFA salt, MeOD) δ 7.41 (s, 1H), 7.40 (d, J = 8.0 Hz, 1H), 7.12 (s, 1H), 6.75 (d, J = 8.0 Hz, 1H), 5.86 (s, 1H), 4.08-3.96 (m, 2H), 3.82 (s, 3H), 3.79-3.51 (m, 3H), 3.49-3.35 (m, 3H), 3.29-3.04 (m, 4H), 2.25-2.04 (m, 2H), 1.84-1.63 (m, 4H), 1.27-1.18 (m, 4H), 0.96-0.84 (m, 4H); 575 [M + H] ⁺ |
| 5 | 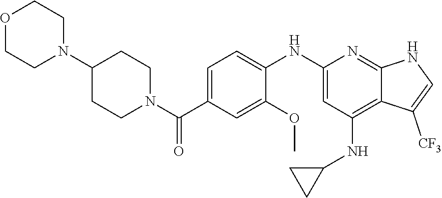 [SMILES: COc1cc(C(=O)N2CCC(N3CCOCC3)CC2)ccc1Nc1cc(NC2CC2)c2c(C(F)(F)F)c[nH]c2n1] | (4-((4-(cyclopropylamino)-3- (trifluoromethyl)-1-((2- (trimethylsilyl)ehotyx)methyl)-1H- pyrrolo[2,3-b]pyridine-6-yl)3- methoxyphenyl) morpholinopiperidine-1-yl) methanone | ¹H NMR (400 MHz, TFA salt, MeOD-d4) δ 7.62 (d, J = 8.18 Hz, 1H), 7.50 (s, 1H), 7.24 (s, 1H), 7.15 (d, J = 7.72 Hz, 1H), 6.42 (s, 1H), 4.10-3.99 (m, 2H), 3.98-3.94 (m, 1H), 3.94 (s, 3H), 3.93-3.79 (m, 2H), 3.72-3.49 (m, 3H), 3.34-3.13 (m, 5H), 2.70-2.65 (m, 1H), 2.40-2.14 (m, 2H), 1.85-1.60 (m, 2H), 1.00-0.94 (m, 2H), 0.76-0.68 (m, 2H); 559 [M + H] ⁺ |

### <Experimental Example 1> Evaluation of Inhibitory Activity of the Compounds According to the Present Invention on Various Kinases

Measuring the enzyme (kinase) selectivity and inhibitory activity of Compound 1 selected from among the example compounds of the present invention was consigned to DiscoverX, and a scanMAX™ Kinase analysis panel was used to carry out the experiment. Here, the concentration of the drug used to treat the enzyme was 1 μM in DMSO. The percentage control (% control) was decided using the following method, and the results are shown in Table 2.

[(Example Compound−Positive Control)/(Negative Control−Positive Control)×100]

Here, the positive control refers to a compound which exhibits a percentage control of 0%, and the negative control represents a 100% percentage control with DMSO. Further, as for the enzyme selectivity of the present invention, if the percentage control for each enzyme was <41% (that is, less than 41%), [the compound] was judged to have activity with regard to such enzyme.

TABLE 2

| Item | Protein Kinase |
|---|---|
| Protein kinases | ALK, ALK(C1156Y), ALK (L1196M), |
| exhibiting 59% | CLK1, CLK2, CLK3, CLK4, cSNK1D, |
| or higher | DYRK1A, DYRK1B, DYRK2, |
| inhibition | GAK, JNK1, LRRK2 (G2019S), LTK, |
| | MYLK, PAK2, PHKG1, PHKG2, STK33 |
| Protein kinases | ABL1-nonphosphorylated, CAMK2D, |
| exhibiting at least | CAMKK2, CHEK2, CSNK1A1, |
| 40% and up to | CSNK1E, ERK5, HUNK, INSR, JAK1 |
| 59% inhibition | (JH2domain-psudokinase), JNK2, JNK3, |
| | LRRK2, MAPKAPK2, PLK4, STK39 |

As shown in Table 2 above, Compound 1 exhibits inhibitory activity against various protein kinases.

### <Experimental Example 2> Evaluation of Lung Cancer Cell Colony Formation Inhibition with Concomitant Treatment Using Compounds of the Present Invention and EGFR Targeting Drug

To evaluate lung cancer cell colony formation inhibition under concomitant treatment using Compound 1 according to the present invention and EGFR targeting drug, two lung cancer cell lines were used to carry out the following experiment.

Specifically, the PC-9 cell line was purchased from the ECACC (European Collection of Authenticated Cell Cultures), and RPMI-1640 with 10% FBS and 1% penicillin streptomycin was used. PC-9GR cells cultured with slow and sustained exposure to Gefitinib (manufactured by Selleckchem) were serially cultured with 1 μmol/L Gefitinib.

Specifically, the H1975 cell line was purchased from the ATCC (American Type Culture Collection, and RPMI-1640 with 10% FBS and 1% penicillin streptomycin was used.

16 to 24 hours prior to treatment with Compound 1 of the present invention, Harmine, Gefitinib, Osimertinib or combinations thereof, 5000 cells were placed in each well of a 12 well plate. The culture medium was replaced every 3 days, distinguishing between wells with and without compounds, and the wells were observed for colony-forming cells starting from 10 days after treatment with the compounds. The remaining colony-forming cells were fixed with methanol (1%) and formaldehyde (1%), then dyed with 0.5% crystal violet before photographing each well.

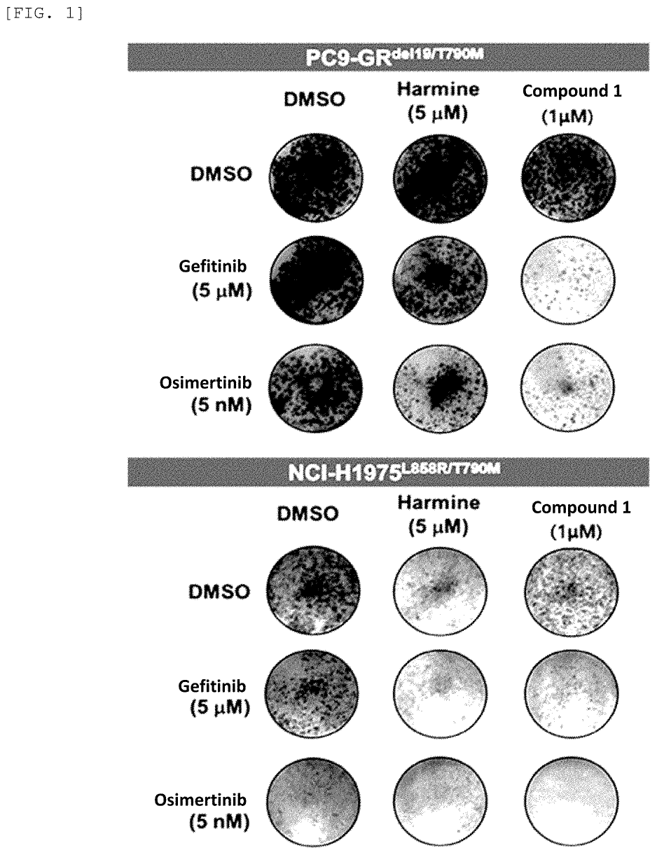

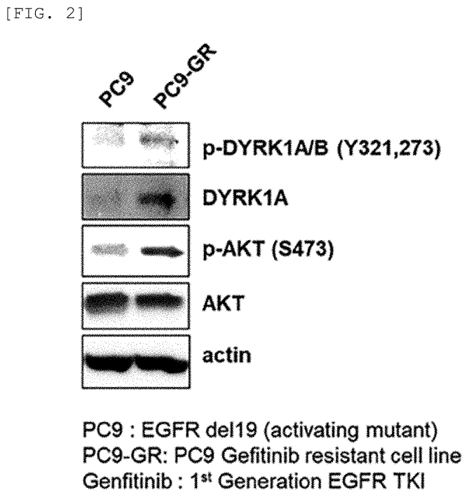

FIG. 1 shows the photographs taken, and represents the results of evaluating color formation inhibition effect of treating EGFR double mutation lung cancer cells concomitantly with the experimental compounds. As shown in FIG. 1, Compound 1 of the present invention, in concomitant treatment with the EGFR TKIs Gefitinib or Osimertinib, was found to substantially inhibit formation of lung cancer cell colonies even when compared against single treatment with the respective compounds or the reference compound Harmine.

### <Experimental Example 3> Comparison of Protein Kinase Expression in Non-Small Cell Lung Cancer Cell Line and Non-Small Cell Lung Cancer Cell Line with Resistance to EGFR Targeting Drug

To compare expression of DYRK1A in a non-small cell lung cancer cell line and a non-small cell lung cancer cell line having Gefitinib resistance, a western blot test was carried out. The cells were treated with 1× RIPA buffer with added protease inhibitor and phosphatase inhibitor, then recovered, followed by lysis for 30 minutes in ice and 15 minutes centrifuging at 14000 rpm and 4° C. The supernatant was isolated, then protein was quantified using a Bradford assay. The western blot test was carried out using the same amount of protein. Reacting for 16 hours at 4° C. with the respective primary antibodies was followed by reaction with secondary antibodies. The results were detected using LAS500.

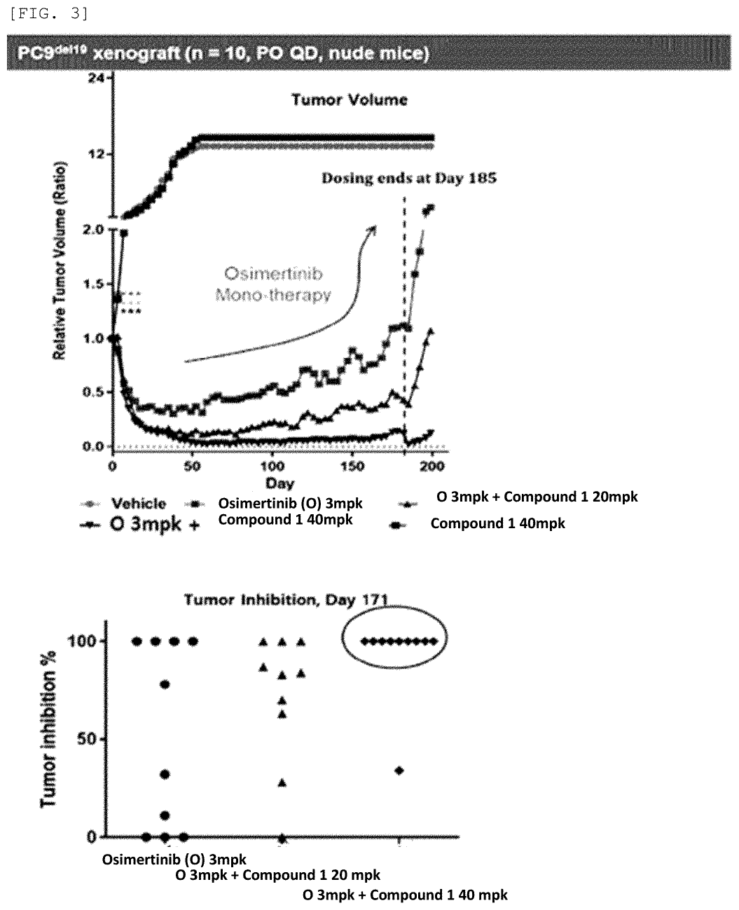

In the results, it was found that DYRK1A/B (Y321, 273) and Akt (S473) were more activated in the cell line with Gefitinib resistance compared to the PC9 human-derived non-small cell lung cancer cell line (FIG. 2). Through the result, it can be expected that protein kinases with increased activity are associated with resistance of non-small cell lung cancer cell lines to EGFR targeting drug (Gefitinib).

### <Experimental Example 4> Evaluation of Anticancer Effect in Mouse Model Concomitantly Treated with Compounds According to the Present Invention and EGFR Targeting Drug

Anticancer effect was examined for cases where a mouse model (Balb/c Nude Mice) with PC-9 lung cancer cells was administered singularly with Compound 1, Gefitinib and Osimertinib, respectively.

The PC-9 cell line was purchased from NanJing Cobioer Biological Technology Co., Ltd. Cells were kept in RPMI-1640 containing 10% FBS, and cultured in a 37° C. humidified cell culture incubator at 5% C₂. All animal experiments were carried out according to the Animal Use and Management Guidelines of Shanghai Medicilon Inc. Under sterile conditions, 2×10⁶ PC-9 tumor cells (0.1 mL, cell:Matrigel®=1:1) was administered to the right waist of each animal by S.C. administration. When the tumors reached a suitable size, 250 mm³, the mice were grouped randomly and treated as shown in Table 3 below. Tumor size and animal body weight were measured twice weekly, and clinical symptoms were recorded daily. The mice will be administered individually according to most recent body weight. The tumors were measured using calipers using two measurements, length (a) and width (b). The volume of the tumors was calculated individually as follows, from the two measured diameters of the tumors: tumor volume (mm³)=(a×b²)/2.

TABLE 3

| | | | Inj. | | | |
|---|---|---|---|---|---|---|
| | | Dose | Vol. | Conc. | | Treatment |
| Group | Treatment | (mg/kg) | (mL/kg) | (mg/mL) | Route | Schedule |
| 1 | 5% DMSO + | (N/A) | 10 | (N/A) | PO | QD × TBD |
| | 5% Tween80 + | | | | | |
| | 90% DDW | | | | | |
| 2 | Gefitinib | 10 | 10 | 1 | PO | QD × TBD |
| 3 | Gefitinib | 10 | 10 | 1 | PO | QD × TBD |
| | Compound 1 | 20 | 10 | 2 | PO | QD × TBD |
| 4 | Gefitinib | 10 | 10 | 1 | PO | QD × TBD |
| | Compound 1 | 40 | 10 | 4 | PO | QD × TBD |
| 5 | Osimertinib | 3 | 10 | 0.3 | PO | QD × TBD |
| 6 | Osimertinib | 3 | 10 | 0.3 | PO | QD × TBD |
| | Compound 1 | 20 | 10 | 2 | PO | QD × TBD |
| 7 | Osimertinib | 3 | 10 | 0.3 | PO | QD × TBD |
| | Compound 1 | 40 | 10 | 4 | PO | QD × TBD |
| 8 | Compound 1 | 40 | 10 | 4 | PO | QD × TBD |

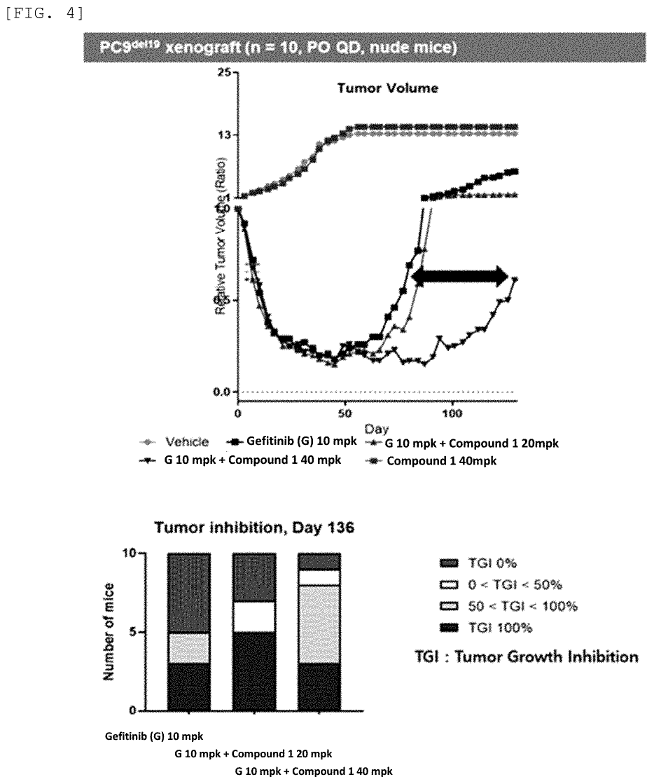

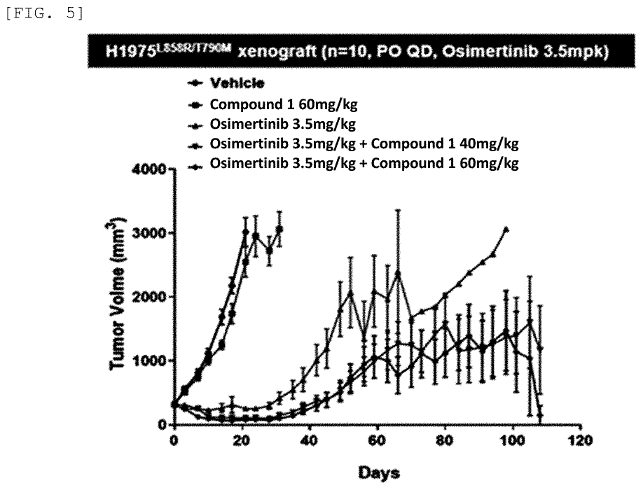

In the results, it was found, as shown in FIG. 3 and FIG. 4, that when compared to cases of singular administration of EGFR targeting drug (Gefitinib or Osimertinib), concomitant use [of these compounds] with Compound 1 of the present invention substantially reduced lung cancer (PC-9) cells, and cancer cell regrowth rates were confirmed to decrease and treatment was sustained. That is, whereas in cases of singular treatment with EGFR targeting drug, early regrowth of cancer cells was observed during treatment, but in cases with treatment with the compound of the present invention, it was found that increase of cancer cells was continually inhibited in a concentration dependent manner. This indicates that concomitant administration of the compound of the present invention also has effect on cancer cell resistance (tolerance) to EGFR targeting drugs. Further, whereas no anticancer effect on the lung cancer cells was observed for singular treatment with Compound 1 of the present invention, concomitant use with an EGFR targeting drug was observed to substantially improve the effect of the EGFR targeting drug.

### <Experimental Example 5> Evaluation of Anticancer Effect of Concomitant Treatment of a Lung Cancer Mouse Model with Compounds According to the Present Invention and EGFR Targeting Drug

Anticancer effect was examined for cases where a mouse model (Balb/c Nude Mice) with NCI-H1975 lung cancer cells was administered singularly with Compound 1, and Osimertinib, respectively.

The NCI-H1975 cell line was purchased from the Institute of Biochemistry and Cell Biology (Shanghai Institutes for Biological Sciences, CAS). Cells were kept in RPMI-1640 containing 10% FBS, and cultured in a 37° C. humidified cell culture incubator at 5% CO₂. All animal experiments were carried out according to the Animal Use and Management Guidelines of Shanghai Medicilon Inc. Under sterile conditions, 2×10⁶ PC-9 tumor cells (0.1 mL, cell:Matrigel®=1:1) was administered to the right waist of each animal by S.C. administration. When the tumors reached a suitable size, 250 mm³, the mice were grouped randomly and treated as shown in Table 3 below. Tumor size and animal body weight were measured twice weekly, and clinical symptoms were recorded daily. The mice will be administered individually according to most recent body weight. The tumors were measured using calipers using two measurements, length (a) and width (b). The volume of the tumors was calculated individually as follows, from the two measured diameters of the tumors: tumor volume (mm³)=(a×b²)/2.

TABLE 4

| | | | Inj. | | | |
|---|---|---|---|---|---|---|
| | | Dose | Vol. | Conc. | | Treatment |
| Group | Treatment | (mg/kg) | (mL/kg) | (mg/mL) | Route | Schedule |
| 1 | 5% DMSO + | (N/A) | 10 | (N/A) | PO | QD × TBD |
| | 5% Tween80 + | | | | | |
| | 90% DDW | | | | | |
| 2 | Compound 1 | 60 | 10 | 6 | PO | QD × TBD |
| 3 | Osimertinib | 3.5 | 10 | 0.4 | PO | QD × TBD |
| 4 | Osimertinib | 3.5 | 10 | 0.35 | PO | QD × TBD |
| | Compound 1 | 40 | 10 | 4 | PO | QD × TBD |
| 5 | Osimertinib | 3.5 | 10 | 0.35 | PO | QD × TBD |
| | Compound 1 | 60 | 10 | 6 | PO | QD × TBD |

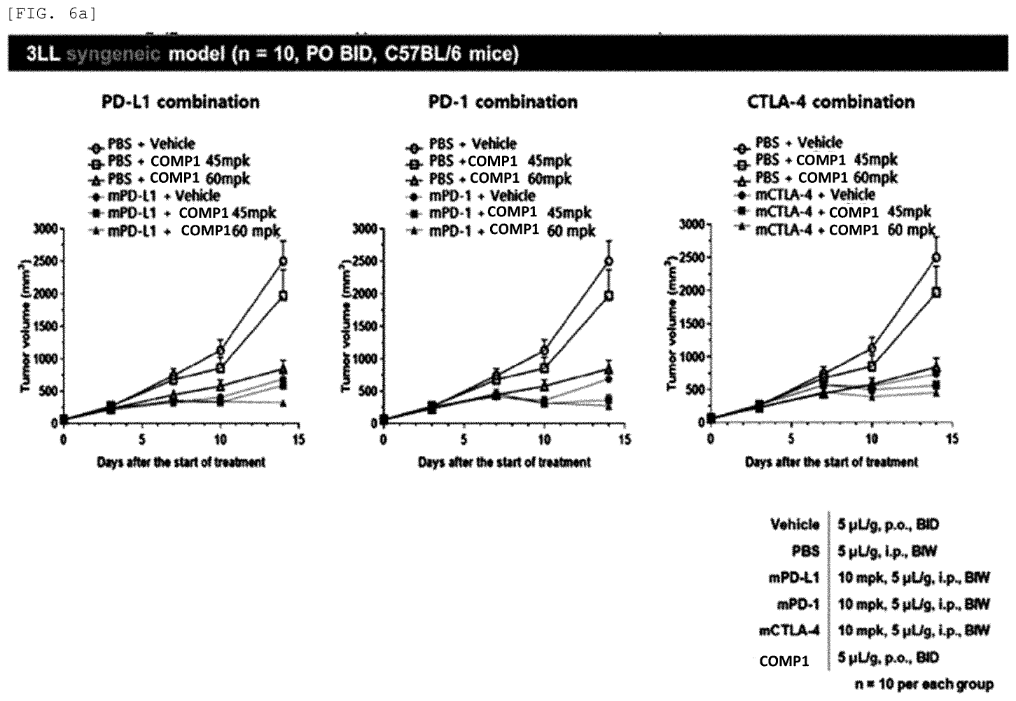

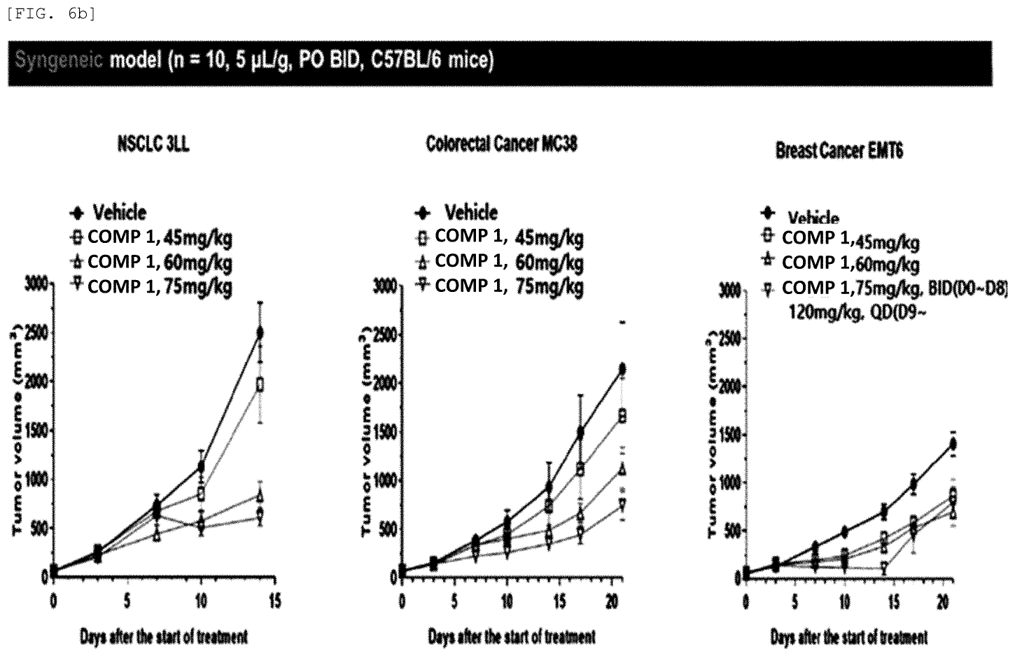

In the results, it was found, as shown in FIG. 5, that when compared to cases of singular administration of EGFR targeting drug (Osimertinib), concomitant use [of the drug] with Compound 1 of the present invention substantially reduced lung cancer (NCI-H1975) cells, and cancer cell regrowth rates were confirmed to decrease and treatment was sustained. That is, whereas in cases of singular treatment with EGFR targeting drug, early regrowth of cancer cells was observed during treatment, but in cases with treatment with the compound of the present invention, it was found that increase of cancer cells was continually inhibited in a concentration dependent manner. This indicates that concomitant administration of the compound of the present invention also has effect on cancer cell resistance (tolerance) to EGFR targeting drugs. Further, whereas no anticancer effect on the lung cancer cells was observed for singular treatment with Compound 1 of the present invention, concomitant use with an EGFR targeting drug was observed to substantially improve the effect of the EGFR targeting drug.

### <Experimental Example 6> Evaluation of Anticancer Effect of Singular Treatment with Compounds According to the Present Invention and Concomitant Treatment with PD-1, PD-L1 and CTLA-4 Antibody in a Lung Cancer, Colon Cancer and Breast Cancer Heteroplastic Transplant Mouse Model

The efficacy of singular use of Compound 1 and concomitant use of the same with PD-1, PD-L1 and CTLA-4 antibody was evaluated in a 3LL lung cancer, MC38 colon cancer and EMT6 breast cancer heteroplastic transplant female C57BL/6 mouse model.

The 3LL cells were cultured in Hams F10:L15=3:7 culture medium, the EMT-6 cells were cultured in Waymouth's MB 752/1 culture medium, and the MC38 cells were cultured in a DMEM culture medium with 10% FBS and 1% penicillin streptomycin, at 37° C. in a 5% CO₂ humidified cell culture incubator.

The mouse PD-1 antibody (BE-0146), PD-L1 antibody (BE-0101), and CTLA-4 antibody (BE-0131) were purchased from BioXcell.

All animal tests were carried out through WuXi AppTec. In all animal tests, Compound 1 was treated at 45, 60, 75 mg/kg by QD and BID, as shown in Table 5 below, and the PD-1, PD-L1 and CTLA-4 antibodies for concomitant effect were treated at 10 mg/kg by IP and BIW.

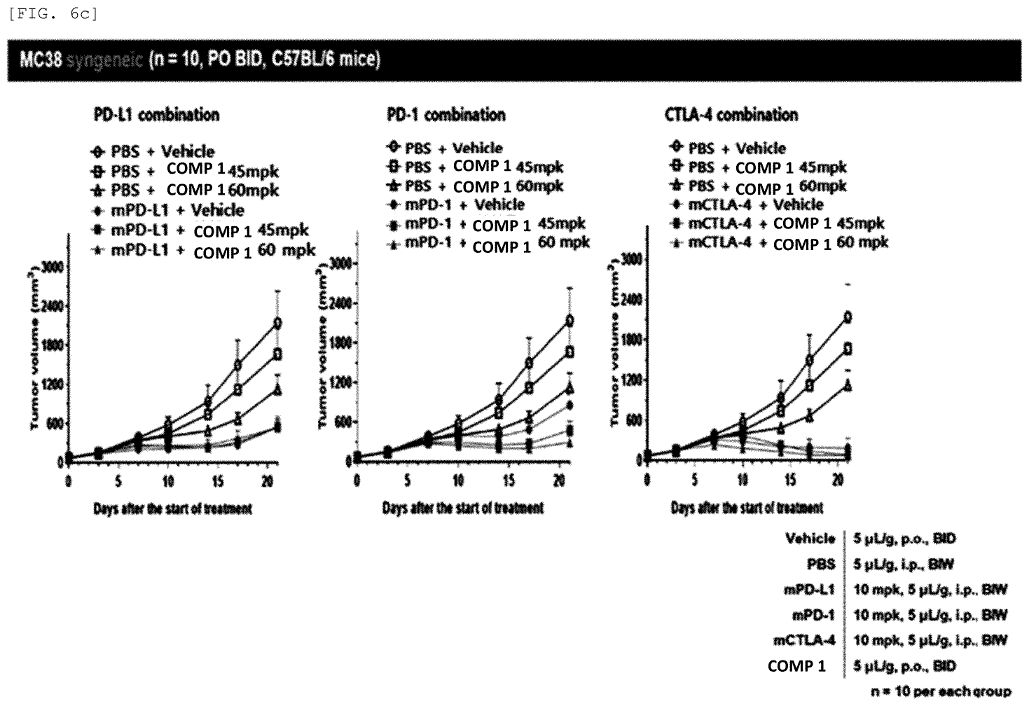

In the results, whereas singular treatment with Compound 1 had insignificant tumor suppression effect, improved concomitant effect with PD-1, PD-L1 and CTLA-4 antibodies was observed (FIG. 6a-6c).

TABLE 5

| | | | | Dosing | | |
|---|---|---|---|---|---|---|
| | | | Dose | Volume³ | Dosing | |
| Group | N² | Treatment | (mg/kg) | (uL/g) | Route | Schedule |
| 1 | 10 | PBS + | — | 6 | ip + | BIW × 5 |
| | | Vehicle | | | po | doses + BID × |
| | | | | | | 17 days |
| 2 | 10 | PBS + | 90 | 6 | ip + | BIW × 5 |
| | | Compound 1 | (45 × 2) | | po | doses + BID × |
| | | | | | | 17 days |
| 3 | 10 | PBS + | 120 | 6 | ip + | BIW × 5 |
| | | Compound 1 | (60 × 2) | | po | doses + BID × |
| | | | | | | 17 days |
| 4 | 10 | PBS + | 150 | 6 | ip + | BIW × 5 |
| | | Compound 1 | (75 × 2) | | po | doses + BID × |
| | | | | | | 17 days |
| 5 | 10 | mPD-L1 + | 10 | 6 | ip + | BIW × 5 |
| | | Vehicle | | | po | doses + BID × |
| | | | | | | 17 days |
| 6 | 10 | mPD-L1 + | 10 + 90 | 6 | ip + | BIW × 5 |
| | | Compound 1 | (45 * 2) | | po | doses + BID × |
| | | | | | | 17 days |
| 7 | 10 | mPD-L1 + | 10 + 120 | 6 | ip + | BIW × 5 |
| | | Compound 1 | (60 * 2) | | po | doses + BID × |
| | | | | | | 17 days |
| 8 | 10 | mPD-L1 + | 10 + 150 | 6 | ip + | BIW × 5 |
| | | Compound 1 | (75 * 2) | | po | doses + BID × |
| | | | | | | 17 days |
| 9 | 10 | mPD-1 + | 10 | 6 | ip + | BIW × 5 |
| | | Vehicle | | | po | doses + BID × |
| | | | | | | 17 days |
| 10 | 10 | mPD-1 + | 10 + 90 | 6 | ip + | BIW × 5 |
| | | Compound 1 | (45 * 2) | | po | doses + BID × |
| | | | | | | 17 days |
| 11 | 10 | mPD-1 + | 10 + 120 | 6 | ip + | BIW × 5 |
| | | Compound 1 | (60 * 2) | | po | doses + BID × |
| | | | | | | 17 days |
| 12 | 10 | mPD-1 + | 10 + 150 | 6 | ip + | BIW × 5 |
| | | Compound 1 | (75 * 2) | | po | doses + BID × |
| | | | | | | 17 days |
| 13 | 10 | mCTLA-4 + | 10 | 6 | ip + | BIW × 5 |
| | | Vehicle | | | po | doses + BID × |
| | | | | | | 17 days |
| 14 | 10 | mCTLA-4 + | 10 + 90 | 6 | ip + | BIW × 5 |
| | | Compound 1 | (45 * 2) | | po | doses + BID × |
| | | | | | | 17 days |
| 15 | 10 | mCTLA-4 + | 10 + 120 | 6 | ip + | BIW × 5 |
| | | Compound 1 | (60 * 2) | | po | doses + BID × |
| | | | | | | 17 days |
| 16 | 10 | mCTLA-4 + | 10 + 150 | 6 | ip + | BIW × 5 |
| | | Compound 1 | (75 * 2) | | po | doses + BID × |
| | | | | | | 17 days |

### <Experimental Example 7> Confirming EGFR Expression Inhibition by Compounds According to the Present Invention in an EGFR Mutant Cell Line and Various Cancer Cell Lines

As an experiment to examine EGFR expression after treating with Compound 1, tests were carried out using Ba/F3 EGFR L858R/T790M/C797S, NCI-H2228 and CAL-27 cell lines and western blot or phosphoarray.

For the Ba/F3 EGFR L858R/T790M/C797S cell line, Ba/F3 naive cells purchased from DSMZ were transfected with EGFR L858R/T790M/C797S plasma DNA using lipofectamine, and a cell line selected using 1 μg/ml puromycin for 2 weeks was used. Here, the culture medium used was RPMI-1640 with 10% FBS and 1% penicillin streptomycin. The NCI-H2228 and CAL-27 cell lines were purchased from ATCC, and for culturing RPMI-1640 with 10% FBS and 1% penicillin streptomycin was used for NCI-H2228, while for CAL-27 DMEM with 10% FBS and 1% penicillin streptomycin was used.

Compound 1 was added to final concentrations of 0.5, 1.2 and 5 μM, followed by culturing for 4 or 24 hours. As needed, culturing was performed for 1 hour with Cyclohexamine at 30 μg/mL concentration, followed by 15 minutes EGF treatment at a concentration of 100 ng/mL. For the phosphoarray experiment, DYRK1a siRNA was reacted with HEK293T cells for 48 hours at 30 nM concentration, confirming inhibition of DYRK1a expression in the cells. These were used as the control for the main experiment. Cal-27 cells were treated for 48 hours with 30 nM DYRK1a siRNA and 2 μM of Compound 1, then proteins were extracted from the cells to carry out the experiment.

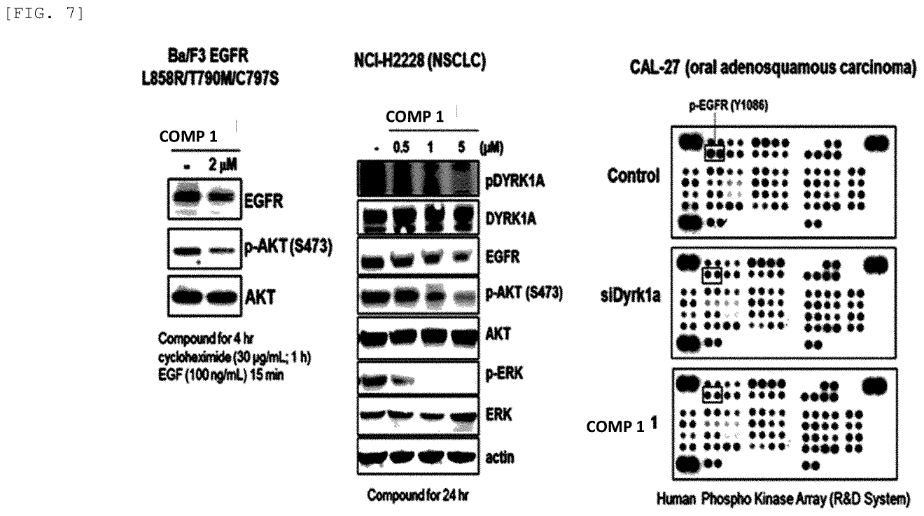

In the results, as shown in FIG. 7, reduced EGFR expression with treatment with Compound 1 was confirmed in Ba/F3 EGFR L858R/T790M/C797S, NCI-H2228 and CAL-27 cells, and reduction of p-AKT (S473) and p-Erk 1/2 (T202/Y204) was confirmed in NCI-H2228 cells. Reduction in expression of siDYRK1A and p-EGFR (Y-1086) was confirmed in CAL-27 cells as well.

### <Experimental Example 8> Effects of Concomitant Use of Compounds According to the Present Invention and EGFR Targeting Drugs in EGFR Mutant Cell Line

To confirm the effect of concomitant use of Compound 1 and Osimertinib in Ba/F3 EGFR L858R/T790M/C797S cells, cells were cultured in a 96 well plate to approximately 2000 cells 16 to 24 hours prior to treatment of the samples. Here, as a control for Compound 1, Harmine, known as a DYRK1A inhibitor, was used. The cells were treated for 72 hours using a serially diluted compound. The cell activity inhibition results for the compounds were measured using celltiterglo.

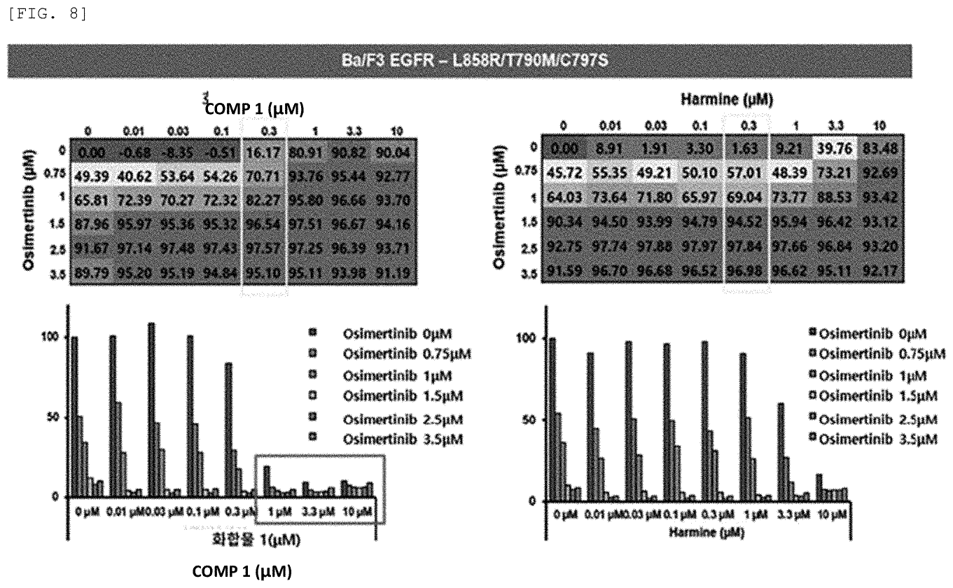

In the results, as shown in FIG. 8, treatment with Compound 1 exhibited improved cell growth inhibition effect compared to treatment with the same amount of Harmine.

What is claimed is:

1. A pharmaceutical composition for the prevention or treatment of cancer, the composition comprising a compound represented by Chemical Formula 1, an isomer of the same or a pharmaceutically acceptable salt of the same; and a targeting drug for at least one of EGFR (epidermal growth factor receptor), PD-1 (programmed cell death protein 1), PD-L1 (programmed death-ligand 1) and CTLA-4 (cytotoxic T-lymphocyte-associated protein 4) as active ingredients: 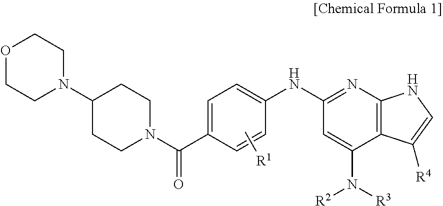
(where, in the Chemical Formula 1,
R1 is C₁-C₃ alkoxy;
R2 and R3 are each independently hydrogen, straight chain or branched chain C¬1-C10 alkyl, or C3-C6 cycloalkyl; and
R4 is haloalkyl.)

2. The pharmaceutical composition for the prevention or treatment of cancer of claim 1, wherein the R1 is methoxy; the R2 is straight chain or branched chain C1-C5 alkyl, or C3-C4 cycloalkyl; the R3 is hydrogen; and R4 is trifluoromethyl.

3. The pharmaceutical composition for the prevention or treatment of cancer of claim 1, wherein the compound represented by Chemical Formula 1 is (4-((4-(ethylamino)-3-(trifluoromethyl)-1H-pyrrolo [2,3-b]pyridine-6-yl)amino)-3-methoxyphenyl) (4-morpholinopiperidine-1-yl) methanone.

4. A pharmaceutical composition for the prevention or treatment of cancer, the composition comprising a compound represented by Chemical Formula 1, an isomer of the same or a pharmaceutically acceptable salt of the same as an active ingredient, characterized in that the composition is concomitantly administered with a targeting drug for at least one of EGFR (epidermal growth factor receptor), PD-1 (programmed cell death protein 1), PD-L1 (programmed death-ligand 1) and CTLA-4 (cytotoxic T-lymphocyte-associated protein 4): 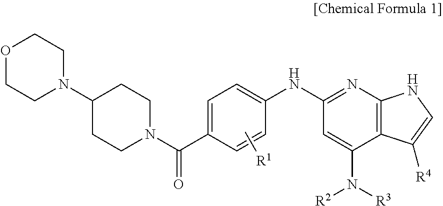
(where, in Chemical Formula 1,
R1 is C1-C3 alkoxy;
R2 and R3 are each independently hydrogen, straight chain or branched chain C¬1-C10 alkyl, or C3-C6 cycloalkyl; and
R4 is haloalkyl.)

5. The pharmaceutical composition for the prevention or treatment of cancer of any one of claim 1 through claim 4, wherein the cancer is at least one selected from a group comprising leukemia, lymphoma, sarcoma, brain cancer, brain tumor, benign stromocytoma, malignant stromocytoma, pituitary cyanoma, meningioma, brain lymphoma, oligodendroglioma, craniopharyngioma, ependymoma, brain stem tumor, head and neck cancer, laryngeal cancer, oropharyngeal cancer, nasal cavity/PNS tumor, nasopharyngeal tumor, salivary gland tumor, hypopharyngeal cancer, thyroid cancer, oral cavity tumor, chest tumor, small cell lung cancer, non-small cell lung cancer, thymoma, thyroid cancer, mediastinal tumor, esophageal tumor, breast cancer, male breast cancer, abdomen-pelvis tumor, stomach cancer, hepatoma, gall bladder cancer, biliary tract tumor, pancreatic cancer, small intestinal tumor, larges intestinal tumor, colorectal cancer, anal cancer, bladder cancer, renal cell carcinoma, male genital cancer, penile cancer, prostatic cancer, female genital cancer, uterine cancer, cervical cancer, endometrial cancer, uterine sarcoma, vaginal cancer, vulva cancer, urethral cancer and skin cancer.

6. The pharmaceutical composition for the prevention or treatment of cancer of any one of claim 1 through claim 4, wherein the cancer is at least one selected from a group comprising brain cancer, breast cancer, cervical cancer, colorectal cancer, bladder cancer, stomach cancer, renal cell carcinoma, head and neck cancer, leukemia, hepatoma, lung cancer, non-small cell lung cancer, small cell lung cancer, lymphoma, gall bladder cancer, ovarian cancer, pancreatic cancer, prostatic cancer, colon cancer, sarcoma, skin cancer, testicular cancer, thyroid cancer and uterine cancer.

7. The pharmaceutical composition for the prevention or treatment of cancer of any one of claim 1 through claim 4, characterized in that the compound, an isomer of the same, or pharmaceutically acceptable salt of the same inhibits the activity of at least one protein kinase selected from a group comprising ALK, ALK (C1156Y), ALK (L1196M), CLK1, CLK2, CLK3, CLK4, CSNK1D, DYRK1A, DYRK1B, DYRK2, GAK, JNK1, LRRK2 (G2019S), LTK, MYLK, PAK2, PHKG1, PHKG2, STK33, ABL1-nonphosphorylated, CAMK2D, CAMKK2, CHEK2, CSNK1A1, CSNK1E, ERK5, HUNK, INSR, JAK1 (JH2domain-pseudokinase), JNK2, JNK3, LRRK2, MAPKAPK2, PLK4, and STK39.

8. The pharmaceutical composition for the prevention or treatment of cancer of any one of claim 1 through claim 4, characterized in that the EGFR targeting drug is at least one selected from a group comprising Brigatinib, CUDC-101, Erlotinib, Gepitinib, Icotinib, Lapatinib, Safitinib, Vandetanib, Baratinib, Tecevatinib, Tirpostin AG 1478, AZD3759, MTKi-327 (JNJ-26483327), Afatinib, Olmutinib (HM61713), Canertinib, CL-387785 (EKI-785), CNX-2006), Dacomitinib, Naquotinib (ASP8273), Neratinib, Osimertinib, PD168393, Pelitinib, Poziotinib, Rociletinib, TAK285, WZ4002, Alitinib (ALS-1306; AST-1306), AV-412 (MP-412), Nazartinib (EGF816), Pyrotinib, Cetuximab and Panitumumab; the PD-1 targeting drug is at least one of Pembrolizumab and Nivolumab; the PD-L1 targeting drug is at least one of Atezolizumab, Avelumab and Duvalumab; and the CTLA-4 targeting drug is Ipilimumab.

9. A kit for prevention or treatment of cancer, the kit comprising a composition comprising: a targeting drug for at least one of EGFR (epidermal growth factor receptor), PD-1 (programmed cell death protein 1), PD-L1 (programmed death-ligand 1) and CTLA-4 (cytotoxic T-lymphocyte-associated protein 4) as active ingredients; and the compound represented by Chemical Formula 1 of any one of claim 1 through claim 3, an isomer of the same or a pharmaceutically acceptable salt of the same.
